SEQUENCE LISTING (8 sequences)

SEQ ID NO: 1 - DNA, 22 bp, Homo sapiens
aaattcggtacatcctcgacgg

SEQ ID NO: 2 - DNA, 23 bp, Homo sapiens
ggaaggttcaggttgttttctgc

SEQ ID NO: 3 - DNA, 23 bp, Homo sapiens
actgagagtgattgagagtggac

SEQ ID NO: 4 - DNA, 21 bp, Homo sapiens
aaccctctgcacccagttttc

SEQ ID NO: 5 - DNA, 20 bp, Homo sapiens
aaggcggccaggatataact

SEQ ID NO: 6 - DNA, 22 bp, Homo sapiens
tacggcctaaggcaggcagttg

SEQ ID NO: 7 - DNA, 25 bp, Homo sapiens
agaaaaacctgccaaatatgatgac

SEQ ID NO: 8 - DNA, 20 bp, Homo sapiens
tgggtgtcgctgttgaagtc

(19) United States
(12) Patent Application Publication
(10) Pub. No.: US 2021/0244773 A1
(43) Pub. Date: Aug. 12, 2021

(54) DENTAL PULP STEM CELLS AND USES THEREOF

(71) Applicants: Leslie Morse, Minneapolis, MN (US); Ricardo Battaglino, Minneapolis, MN (US)

(72) Inventors: Leslie Morse, Minneapolis, MN (US); Ricardo Battaglino, Minneapolis, MN (US)

(21) Appl. No.: 17/049,372

(22) PCT Filed: May 1, 2019
(86) PCT No.: PCT/US2019/030197

§ 371 Date: Oct. 21, 2020

Related U.S. Application Data
(60) Provisional application No. 62/665,757, filed on May 2, 2018.

(51) Int. Cl. A61K 35/545; C12N 5/0775; A61K 9/00

(52) U.S. Cl. CPC A61K 35/545; A61K 9/0019; C12N 5/0664

(57) ABSTRACT

The disclosure relates to dental pulp stem cells, method of preparing dental pulp stem cells, and methods of treating a patient with nervous tissue injuries by using dental pulp stem cells.

### CROSS-REFERENCE TO RELATED APPLICATIONS

This application claims priority to U.S. Application Ser. No. 62/665,757, filed on May 2, 2018. The disclosure of the prior application is considered part of the disclosure of this application, and is incorporated in its entirety into this application.

### FIELD

The disclosure relates to dental pulp stem cells and methods of preparing such cells. The disclosure also relates to the use of the cells for treating nervous tissue damage, such as a spinal cord injury.

### BACKGROUND

Damage to the central nervous system, such as spinal cord injury, is serious and extremely debilitating. Serious damage can cause quadriplegia or paraplegia. A number of clinical trials directed at the use of stem cells to treat spinal cord injuries are underway, including trials based on the use of oligodendrocyte progenitor cells, olfactory en-sheathing cells or mesenchymal cells. All trials have reported favorable safety profiles, but none has reported improvements in function, and have thus failed to demonstrate efficacy.

Accordingly, there is a need in the art for improved ways of treating nervous tissue damage, particularly using dental pulp stem cells.

### SUMMARY

In one aspect the invention comprises a pharmaceutical formulation comprising dental pulp stem cells and a pharmaceutically acceptable carrier, wherein the dental pulp stem cells are derived from dental pulp from a permanent tooth.

In another aspect the dental pulp stem cells are derived from dental pulp that has not been exposed to an exogenous proteolytic enzyme.

In another aspect the dental pulp stem cells do not comprise exogenous DNA.

In another aspect the pharmaceutical formulation comprising the dental pulp stem cells comprises at least 10⁶, 2×10⁶, 3×10⁶, 4×10⁶, 5×10⁶, 6×10⁶, 7×10⁶, 8×10⁶, 9×10⁶, or 10⁷ dental pulp stem cells.

In a preferred aspect the pharmaceutical formulation comprises 5×10⁶ dental pulp stem cells.

In one aspect the invention comprises an injection device comprising the pharmaceutical formulation.

In some aspects the injection device comprising the pharmaceutical formulation is a syringe.

In one aspect the invention comprises a method of preparing dental pulp stem cells, comprising:

culturing dental pulp extracted from a permanent tooth, in a first culture medium until the dental pulp yields outgrowing dental pulp stem cells;

culturing the dental pulp from the first culture medium in a second culture medium until the dental pulp yields outgrowing dental pulp stem cells;

optionally, culturing the dental pulp in a third culture medium until the dental pulp yields outgrowing dental pulp stem cells;

wherein the dental pulp is not exposed to an exogenous proteolytic enzyme and the dental pulp stem cells lack exogenous DNA.

In another aspect, the dental pulp stem cells are fibroblast-like.

In another aspect the invention comprises a method of preparing dental pulp stem cells, wherein each culturing step is performed for 3-4 days.

In another aspect the invention comprises a method of preparing dental pulp stem cells, wherein each culturing step comprises culturing in Dulbecco's Modified Eagle's Medium/F-12/Fetal Bovine Serum.

In another aspect the invention comprises a method of preparing dental pulp stem cells, wherein after the final culturing step, the culture medium comprises 10⁶-10⁷ dental pulp stem cells.

In another aspect the invention comprises a method of preparing dental pulp stem cells wherein the dental pulp stem cells in the first, second, and/or third culture medium are grown to 30-80 percent, or 50-70 percent, confluence. The dental pulp stem cells may be passaged and expanded up to three times. The expanded dental pulp stem cells may be used to prepare a pharmaceutical composition.

In another aspect the invention comprises a method of preparing dental pulp stem cells wherein the dental pulp stem cells may be treated with an enzyme prior to subsequent passage or harvest.

In another aspect the invention comprises a method of making dental pulp stem cells, further comprising after the final culturing step, suspending the dental pulp stem cells in a pharmaceutically acceptable carrier.

In another aspect the invention further comprising loading dental pulp stem cells suspended in a pharmaceutically acceptable carrier into an injection device.

In another aspect the invention the injection device containing the dental pulp stem cells is a syringe.

In another aspect the invention the method for making dental pulp stem cells comprises culturing dental pulp stem cells from dental pulp extracted from a permanent tooth, wherein the tooth is a molar.

In one aspect the invention comprises a method of treating a spinal cord injury comprising a spinal cord lesion in a patient in need thereof, comprising:

administering dental pulp stem cells to the patient, wherein the dental pulp stem cells are extracted in a tooth-preserving root canal procedure from a permanent tooth of the patient, wherein the dental pulp from which the dental pulp stem cells are derived has not been exposed to an exogenous proteolytic enzyme, and wherein the dental pulp stem cells do not comprise exogenous DNA.

In another aspect the invention comprises a method of treating a spinal cord injury comprising a spinal cord lesion in a patient in need thereof, comprising:

administering dental pulp stem cells to the patient, wherein the dental pulp stem cells are extracted in a tooth-preserving root canal procedure from a molar from the patient, wherein the dental pulp from which the dental pulp stem cells are derived has not been exposed to an exogenous proteolytic enzyme, and wherein the dental pulp stem cells do not comprise exogenous DNA.

In another aspect the invention comprises a method of treating a spinal cord injury comprising a spinal cord lesion in a patient in need thereof, wherein the dental pulp stem cells are administered 45-60 days after the patient suffers the spinal cord lesion.

In another aspect the invention comprises a method of treating a spinal cord injury comprising administering 10⁶-10⁷ cells per cubic centimeter per lesion volume in 0.5-3 mL of a pharmaceutically acceptable carrier.

In another aspect the invention comprises a method of treating a spinal cord injury with dental pulp stem cells, wherein the administering step comprises directly injecting the dental pulp stem cells into the spinal cord of the patient.

In another aspect the invention comprises a method of treating a spinal cord injury by injection of human dental pulp stem cells, wherein the injection is at the caudal aspect of the spinal cord lesion.

In another aspect the invention comprises a method of treating a spinal cord injury by injection of human dental pulp stem cells, wherein, wherein the spinal cord is exposed.

In another aspect the invention comprises a method of treating a spinal cord injury by injection of human dental pulp stem cells, wherein the spinal cord is exposed by performing a posterior laminectomy.

In another aspect the invention comprises a method of treating a spinal cord injury by administering human dental pulp stem cells, further comprising before the administering step, performing cord untethering and expansion duraplasty on the patient.

In another aspect the invention comprises a method of treating a spinal cord injury in a patient by administering human dental pulp stem cells, wherein the patient's spinal cord injury is categorized as T3-10 ASIA Impairment Scale (AIS) A or B, due to traumatic spinal cord injury with a single spinal cord lesion.

In another aspect the invention comprises a method of treating a spinal cord injury in a patient by administering human dental pulp stem cells, wherein the patient suffers the spinal cord injury within 3 months to 1 year of the administering step, or more than 1 year before the administering step.

In another aspect the invention comprises a method of treating a spinal cord injury in a patient by administering human dental pulp stem cells, wherein the spinal cord injury is a cervical or thoracic motor complete injury categorized as MS A or B at level C4-C8, or is a motor incomplete injury categorized as AIS C or D.

In another aspect the invention comprises a method of treating a spinal cord injury in a patient by administering human dental pulp stem cells, and an anti-inflammatory agent or a suppressor of spinal cord injury-induced autoimmunity, to the patient. The anti-inflammatory agent may be a resolvin or a lipoxin. The anti-inflammatory agent may be a TNF-alpha inhibitor. The TNF-alpha inhibitor may be adalimumab, certolizumab pegol, etanercept, golimumab, or infliximab. The suppressor of spinal cord injury-induced autoimmunity may be belimumab, atacicept, or blisibimod. One or more anti-inflammatory agents or suppressors of spinal cord injury-induced autoimmunity may be administered singly or in combination, and may be administered separately or together with the dental pulp stem cells.

### DESCRIPTION OF THE DRAWINGS

FIG. 1. Schematic of method for passaging dental pulp multiple times (along left vertical diagonal) and production and passaging of dental pulp stem cells (from left to right along each horizontal). DP=dental pulp, IDPSC=induced dental pulp stem cells, days indicate days from passage 0 (P0), P1, P2, P3 indicates passage 1, passage 2, passage 3, respectively.

Estimated typical cell numbers are indicated at each stage.

FIG. 2. Plot of BBB score (0-21 on vertical axis) over time (as indicated)) of three different cohorts of age matched male Sprague-Dawley (SD) rats. The first cohort (▴) consisted of 6 rats were subjected to laminectomy only without contusion injury and served as a positive control cohort (SHAM SCI). A second cohort (♦) consisting of 9 rats with spinal contusions were subjected to laminectomy only served as a negative control cohort (SEVERE SCI). The third cohort (▪) consisted of 11 rats with spinal contusions subjected to laminectomy and were further treated with human dental pulp stem cells served as the experimental cohort SEVERE SCI PLUS DPSC).

FIG. 3. Plot of abundance of dental pulp stem cells expressing doublecortin, tubulin, and NeuN (neural markers) and Oct 3/4 (stem cell marker) under neuro-inductive conditions by flow cytometry after 21 days of culture. The relative abundance of cells expressing each indicated marker under each condition are shown at the bottom right of each panel.

FIG. 4A-C. Plot of gene expression (RQ values on vertical axis) of dental pulp stem cells treated with different concentrations of TNF-alpha (0, 1, 10, 25 and 100 ng/ml on horizontal axis) for 24 hours prior to analysis. FIG. 2A is expression of IL-6. FIG. 2B is expression of IL-8. FIG. 2C is IL-1β. In each Figure the box contains 50% of the data points and the middle line of the box represents the median. The tips of the projecting bars show minimum and maximum values, n=7. (*) indicates Mann-Whitney, p>0.05. Values that are not statistically different are indicated by using the same letter.

FIG. 5. Plot of the number of cells expressing Oct 3/4, Doublecortin, and β-III tubulin expression by dental pulp stem cells submitted to neuronal differentiation in DMEM/F12 for 21 days in the presence (25 ng/ml) or absence of TNF-alpha (Differentiated DPSC+TNF-alpha (25 ng/ml) and Differentiated DPSC, respectively). Undifferentiated cells under similar conditions are also plotted (Undifferentiated DPSC). The error bars show minimum and maximum values, n=7, (*) indicates Mann-Whitney, p>0.05.

FIG. 6. Flow cytometry analysis of neuronal differentiation of dental pulp stem cells in the presence of TNF-alpha with or without resolving or lipoxin treatment. Relative numbers (percent of total cells) of differentiated cells is plotted along the vertical axis. A significant decrease in β-III tubulin expression occurred in the presence of TNF-alpha. This was mitigated by lipoxin and resolving treatment at both 10 and 100 nM. (*) indicates p<0.05.

### DETAILED DESCRIPTION

Dental pulp stem cells derived from an autologous, permanent tooth of a patient were found to be effective for treating nervous tissue damage, such as a spinal cord injury. Without being bound by theory, the disclosed dental pulp stem cells, having an embryonic origin in the neural crest, appear to possess a heightened capacity for neural differentiation as compared to other sources of self-derived stem cells such as mesenchymal- or adipose stem cells. Further, autologous dental pulp stem cells have a low potential for triggering rejection and thus minimize the need for immunosuppression. The cells may also be rapidly expanded for transplantation as described herein.

Dental pulp stem cells enjoy several other advantages over current stem cell sources. Dental pulp stem cells avoid the ethical issues associated with embryonic stem cells, in contrast to cord-derived stem cells, the source material is readily available, and can be obtained with minimally invasive procedures relative to stem cells derived from bone marrow harvest.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise. The word “about” in association with a numeric value denotes a reasonable approximation of that value. In certain cases “about” may be construed as being within as much as 10% of the specific value with which it is associated. For example, the phrase “about 100” would encompass any value between 90 and 110.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

“Treatment” or “treating,” when referring to protection of an animal from a disease, means preventing, suppressing, repressing, or completely eliminating the disease. Preventing the disease involves administering a composition of the disclosure to an animal prior to onset of the disease. Suppressing the disease involves administering a composition of the disclosure to an animal after induction of the disease but before its clinical appearance. Repressing the disease involves administering a composition of the disclosure to an animal after clinical appearance of the disease.

### 2. Dental Pulp Stem Cells

Provided herein is a dental pulp stem cell. The dental pulp stem cell may be grown from a dental pulp of a deciduous or permanent tooth. As used herein, the term “permanent tooth” or “adult tooth” means a tooth that is not deciduous. In particular, the dental pulp stem cell may be autologous and the dental pulp may be from a permanent tooth. The tooth may be a molar, and may be free of caries and decay. The dental pulp may not have been exposed to an exogenous enzyme. An enzyme may be exogenous if it is contained in a medium to which the dental pulp stem cell is exposed. The enzyme may be a proteolytic enzyme. Proteolytic enzymes are commonly used to dissociate cultured cells so that they can be transferred. The dental pulp may not be treated with an exogenous proteolytic enzyme. The proteolytic enzyme may be a trypsin, a collagenase, an elastase, a pancreatin, a hyaluronidase, or a papain. The dental pulp stem cell may have not been genetically manipulated or altered, such as through transfection of an exogenous nucleic acid, which may be RNA or DNA.

The dental pulp stem cell may be intended for use in transplantation into a patient, and may be autologous to the patient. The patient may also be a close familial blood relative of the donor such a child or grand-child or the like, in which case the method is less dependent on the age of the recipient. For example, if the patient is elderly, dental pulp from which dental pulp stem cells are grown may be from a permanent or deciduous tooth of a close familial relative, and the dental pulp stem cells may be administered to the patient, thereby extending the benefits of the compositions and methods disclosed herein to patients lacking suitable autogenous material. Likewise, if the patient is a young child or infant lacking permanent teeth, dental pulp stem cells grown from dental pulp of a permanent tooth of a close familial relative may be administered to the patient, thereby extending the benefits of the compositions and methods disclosed herein to patients lacking suitable autogenous material. In other situations the dental pulp stem cells may be cryo-preserved for future use as described herein. The patient may be any mammal that has teeth, and may be a pig, a horse, a cow, a sheep, a goat, a dog, a cat, a monkey, an ape, or a human. The tooth or dental pulp thereof may be freshly extracted. The tooth extraction may be performed using any appropriate method known in the art, such as by a root canal procedure.

The dental pulp from which the dental pulp stem cells are grown may have been transferred every 2-5 days, or every 3 or 4 days. The container may be a cell culture dish or plate. The dental pulp may have been transferred to a new container 1, 2, 3, 4, or 5 times. The dental pulp may have been cultured for 10, 11, 12, 13, 14, 15, 16, 17, 18, 19, 20, 21, 22, 23, or 24 days or a range thereof. The dental pulp may have been cultured for 14-21 days.

The dental pulp may be cultured until it yields outgrowing dental pulp stem cells. The dental pulp stem cells may be undifferentiated cells with low cytoplasm to nucleus ratio. The dental pulp stem cells may also be fibroblast-like cells, which may comprise an elongate cell body with distinct pseudopods. Such cells may be characterized by high expression of CD44 and low expression of NeuN. The dental pulp stem cells may comprise a mixed population of undifferentiated cells with low cytoplasm to nucleus ratio and fibroblast-like cells with pseudopods. Such cells may be distinguished by a high expression of CD44 and low expression of NeuN.

The dental pulp stem cells may have been passaged and expanded 1, 2, 3, 4, or 5 times, preferably no more than 1, 2, or 3 times. The dental pulp stem cells may have been treated with a proteolytic enzyme described herein being passaged. For example, the dental pulp stem cells may be trypsinized before passage. The dental pulp stem cells may also be treated with a proteolytic enzyme before being harvested. The dental pulp stem cells may be cultured until about 30-80 percent, or about 50-70% confluence, before being passaged or harvested.

A pharmaceutical formulation may comprise the dental pulp stem cells. The formulation may comprise a pharmaceutically acceptable carrier, which may be sterile normal saline. The basic pharmaceutically acceptable carrier may be supplemented with an anti-inflammatory agent or suppressor of spinal cord injury-induced autoimmunity as described herein. The formulations may comprise about 10⁶, 2×10⁶, 3×10⁶, 4×10⁶, 5×10⁶, 6×10⁶, 7×10⁶, 8×10⁶, 9×10⁶, or 10⁷ dental pulp stem cells or a range thereof. The formulation may range from about 10⁶ to 10⁷ dental pulp stem cells. The formulation may also comprise about 5×10⁶ cells. The formulation may have a volume of about 0.5, 1.0, 1.5, 2.0, 2.5, or 3.0 mL.

An injection device may comprise the dental pulp stem cells or the pharmaceutical formulation. The injection device may comprise a syringe, a needle, a catheter, or any other appropriate means known in the art for administering the cells.

### 3. Methods of Preparing Stem Cells

Provided herein is a method of preparing the dental pulp stem cells from the dental pulp. The method may comprise extracting or harvesting dental pulp from a tooth of a patient, which in particular may be a permanent tooth. The tooth may be free of caries and/or decay. The tooth may be harvested from the patient within 0-3 weeks, particularly within 2 weeks, of the patient having suffered from nervous tissue damage as described herein. When the nervous tissue damage is a spinal cord injury, the spinal cord injury may be acute. The spinal cord injury may comprise a single cord lesion, which may be diagnosed with magnetic resonance imaging. The tooth may be harvested by any method known in the art, such as by a root canal procedure.

Extracting the pulp from the harvested tooth may comprise cutting the tooth, which may be longitudinally. The cutting may be performed with a circular saw. The extracting may also be performed on the patient within 3 months, within 3-12 months, or more than 12 months after the patient suffers from the nervous tissue damage.

The method may further comprise culturing the extracted pulp, which may be freshly extracted (particularly within a few hours or at most, within a few days), in a culture medium. The culture medium may be in a dish, a plate a tube or any other appropriate means known in the art. The culture medium may by any suitable cell culture medium known in the art, and may comprise Dulbecco's Modified Eagle's Medium (DMEM) or DMEM/F-12, and may further comprise Fetal Bovine Serum. The suitable cell culture medium may be supplemented with antibiotics including but not limited to penicillin and streptomycin. The suitable cell culture medium may be supplemented with amino acids including but not limited to glutamine, as well as histidine, isoleucine, leucine, lysine, methionine, phenylalanine, threonine, tryptophan, and valine. The culturing may be performed under sterile conditions. The culturing may be performed for 4, 5, 6, 7, 8, 9 or 10 days or a range thereof. The culturing may be performed until the dental pulp yields dental pulp stem cells as described herein.

After the dental pulp stem cells outgrow from the dental pulp, the method may further comprise transferring the dental pulp to a new culture medium. In one embodiment, dental pulp is transferred to establish a new passage 0 culture. The dental pulp may be transferred (passaged) multiple times to generate multiple passage 0 cultures. The dental pulp may be passaged up to 1 2, 3, 4, 5 or 6 times, and in particular, no more than 1, 2, 3 or 4 times. The dental pulp may never be exposed to a proteolytic enzyme during the course of the methods described herein.

The dental pulp stem cells from the dental pulp may also be transferred (passaged) to a new culture medium and expanded multiple times to generate large numbers of such cells, which may be harvested for use as described herein. The dental pulp stem cells may be passaged up to 1, 2, 3, 4 or 5 times, and in particular, no more than 1, 2, or 3 times. The dental pulp stem cells produced by these methods may be treated with proteolytic enzymes to facilitate their passage and their use in such treatments. The dental pulp stem cells may be passaged and expanded until the total number of dental pulp stem cells of the dental pulp is about 10⁶, 2×10⁶, 3×10⁶, 4×10⁶, 5×10⁶, 6×10⁶, 7×10⁶, 8×10⁶, 9×10⁶, or 10⁷, or a range thereof. The total number of dental pulp stem cells may be about 10⁶ to 10⁷. The total number may also be about 5×10⁶ cells

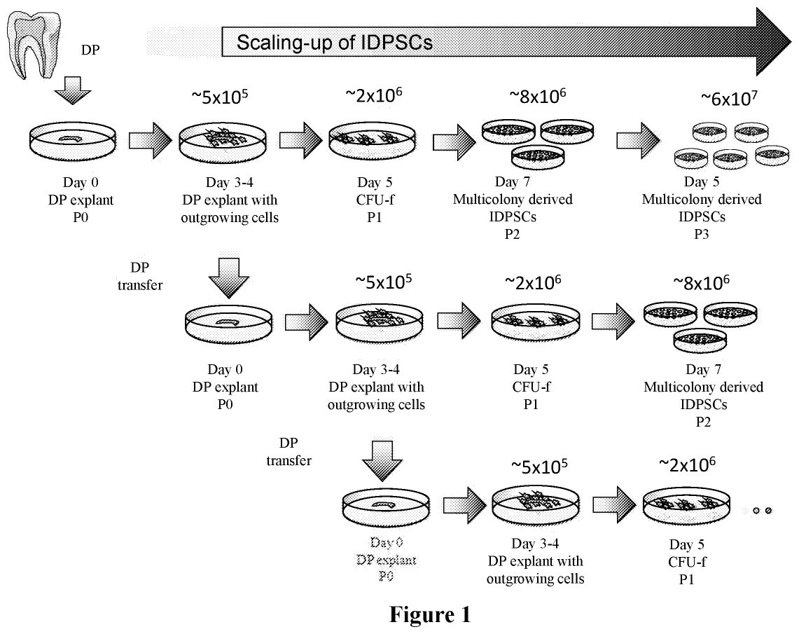

The transferred dental pulp or dental pulp stem cells may be cultured for 2, 3, 4, 5, or 6 days, and particularly 3 or 4 days. The method may comprise culturing the dental pulp or dental pulp stem cells for a total of 12, 13, 14, 15, 16, 17, 18, 19, 20, 21, 22, 23, 24, 25, 26, 27, 27, 28, 29, 30, 31, 32, 33 or 34 days or a range thereof. The method may comprise culturing the dental pulp or dental pulp stem cells for 14-21 days. The method may comprise culturing the dental pulp or dental pulp stem cells for as few as 4 days or more than 34 days. Each culturing step of dental pulp may yield outgrowing dental pulp stem cells. The dental pulp stem cells may be cryopreserved. The method may comprise not exposing the dental pulp to an exogenous enzyme as described herein, such as a proteolytic enzyme. However the method may comprise exposing dental pulp stem cells to an exogenous enzyme as described herein, such as a proteolytic enzyme, before being passaged or harvested. A schematic representation of the method for passaging dental pulp and scaling up production of dental pulp stem cells as described herein is presented in FIG. 1.

Methods of Treating Nervous Tissue Damage

Provided herein is a method of treating nervous tissue damage in a patient in need thereof. The method may comprise administering dental pulp stem cells to the patient. The dental pulp stem cells may be as described herein, may be formulated as described herein, and may be prepared using a process described herein. The dental pulp stem cells may be autologous to the patient and may be from a permanent tooth. The dental pulp stem cells may also be from a blood relative of the patient as described herein and may be from a deciduous tooth of a close blood relative of the patient. The dental pulp stem cells may have been cryopreserved and thawed prior to administration to the patient. The nervous tissue damage may be to the brain or peripheral nerves. The nervous system damage may be due to a stroke or traumatic brain injury. The nervous tissue injury may be a spinal cord injury, brain damage or a peripheral nerve injury, or may be caused by a neurological disorder or disease. The nervous tissue damage may cause paralysis. The spinal cord injury may be acute, and may comprise a lesion, which may be a single lesion. The treatment may promote muscle strength and/or sensation, and may partially or fully restore muscle strength and/or sensation.

The patient may be an adult, and may be at least 18 years old, 18-50 years old, or over 50 years old. The patient may also be less than 18 years old. The patient may have a subacute (3 months-1 year) or a chronic (more than 1 year post-injury) cervical or thoracic motor complete injury. The spinal cord injury may be categorized as T3-10 ASIA Impairment Scale (AIS) A or B. The spinal cord injury may be categorized as AIS A or B at C4-8, or a motor complete injury, which may be categorized as AIS C or D.

The method may comprise administering the dental pulp stem cells 21-60 days, particularly 45-60 days, after the patient suffers the nervous tissue damage. The cells may be administered to the patient after inflammation and/or autoimmune reaction levels triggered by the damage have subsided. The cells may be administered directly into the nervous tissue, which may be exposed. The administration may be performed under general anesthesia. The administration may be an injection. The injection may be intrathecal, intraspinal or systemic.

The method may comprise exposing the spinal cord by posterior laminectomy. The method may also comprise directly visualizing the spinal cord injury, and further confirming the injury by intraoperative ultrasound. The cells may be administered at the caudal aspect of the lesion epicenter. The method may also comprise cord untethering and expansion duraplasty, which may treat arachnoid scarring, before administering the dental pulp stem cells.

The method may also comprise extracting dental pulp stem cells as described herein, and may also comprise culturing dental pulp stem cells as described herein. The method may comprise extracting the dental pulp from the patient within 0-3 weeks, particularly within 2 weeks, after the patient suffers the nervous tissue damage. The extracting may also be performed within 3 months, within 3-12 months, or more than 12 months after the patient suffers the nervous tissue damage. The method may comprise suspending the cultured dental pulp stemcells in the formulation as described herein, loading the suspended dental pulp stem cells into an injection as described herein, and administering the dental pulp stem cells to the patient. The dental pulp stem cells may be administered as a single dose. The administration may be of about 10⁶, 2×10⁶, 3×10⁶, 4×10⁶, 5×10⁶, 6×10⁶, 7×10⁶, 8×10⁶, 9×10⁶, or 10⁷ dental pulp stem cells per cm³ per lesion volume suspended in about 0.5, 1.0, 1.5, 2.0, 2.5, or 3.0 mL, particularly 1.0 mL.

Stem cell therapy may be compromised by inflammation or autoimmunity provoked by spinal cord injury or other nervous system damage. For at least this reason the method may also comprise administering an anti-inflammatory agent to the patient. The anti-inflammatory agent may be administered systemically, which may be oral, parenteral, or intraspinal, or through any other appropriate means known in the art. The anti-inflammatory agent may maximize the differentiation potential or growth factor production of the dental pulp stem cells. The anti-inflammatory agent may be a TNF-alpha inhibitor. The anti-inflammatory agent may comprise adalimumab and biosimilars thereof, certolizumab pegol, etanercept and biosimilars thereof, golimumab, or infliximab. The anti-inflammatory agent may comprise a resolvin, a lipoxin or other small molecule anti-inflammatory agent. The method may also comprise administering a suppressor of spinal cord injury-induced autoimmunity to the patient. The suppressor of spinal cord injury-induced autoimmunity may comprise belimumab, atacicept, or blisibimod. The method may comprise administration of anti-inflammatory drugs or suppressors of spinal cord injury-induced autoimmunity prior to, concomitant with or after administration of dental pulp stem cells to the patient. The anti-inflammatory agent and/or suppressor of spinal cord injury-induced autoimmunity may be administered orally or parenterally, such as by injection or infusion as described herein, or by any appropriate means known in the art. Anti-inflammatory agents and suppressors of spinal cord injury-induced autoimmunity may be administered singly or in combination. Anti-inflammatory agents and suppressors of spinal cord injury-induced autoimmunity may be administered prior to, concomitant with, or after administration of dental pulp stem cells. The method may also comprise providing to the patient a neurorestorative therapy, such as body-weight supported treadmill training, exoskeleton-assisted gait training, or spinal cord stimulation.

The disclosure has multiple aspects, illustrated by the following non-limiting examples.

### Example 1

### Dental Pulp Stem Cells Promote Motor Recovery in Rats with Contusion Spinal Cord Injury

This Example examines the potential of dental pulp stem cells for treatment of spinal cord injury in a rat model.

Dental pulp were isolated from molar teeth of healthy human donors (10-38 yrs. of age) extracted during routine dental care. Pulp was then cultured in basal medium, DMEM/F12+15% FBS supplemented with 100 U/mL non-essential amino acids (Gibco Life Technologies), 100 U/mL penicillin and streptomycin, and glutamine (Gibco Life Technologies) until fibroblast-like dental pulp stem cells were observed. Dental pulp stem cells were passaged after reaching 80% confluence and passage 4 was used for all experiments. Dental pulp stem cells were plated in duplicate with 2×10⁴ cells/well. For flow cytometry experiments, neural differentiation was induced by replacing DMEM/F12+15% FBS culture media with neural differentiation medium (NeuroCult NS-A Differentiation Kit-Human, StemCell Technologies Inc., Tukwila, Wash., USA) 48 hours after plating. Dental pulp stem cells were cultured under neuronal induction conditions for up to 21 days with culture media changed twice a week.

Expression of stem cell and neuronal cell markers was determined by flow cytometry. Cells were fixed with 4% paraformaldehyde for 30 minutes, then permeabilized with 0.1% Triton X-100 for 10 min, and subsequently blocked with TBST in 5% BSA for 20 minutes. Cells were incubated for 1 hour at room temperature with the following primary antibodies against stem cell markers: anti-CD44 (Abcam, 1:30) and anti-OCT4 (Abcam, 1:200) and neuronal markers: anti-nestin (Abcam, 1:100), anti-doublecortin (Abcam, 1:50), anti-β III-tubulin (Abcam, 1:100), and rabbit anti-NeuN (Abcam, 1:100). Fluorescence was achieved by incubating with secondary antibodies for 1 hour at room temperature in a dark room. Cells were then subjected to flow cytometry analysis and the percentage of cells expressing each marker was calculated.

At 21 days of neuronal induction, there was a significant reduction in the percentage of cells expressing the stem cell markers CD44 (57% reduction, p<0.05) and OCT 3/4 (42% reduction, p<0.005). Concomitantly, significantly more cells expressed the early neuronal markers nestin (300% increase, p<0.005) and doublecortin (62% increase, p<0.05), the intermediate neuronal marker β III-tubulin (228% increase, p<0.005), and the late neuronal marker NeuN (418% increase, p<0.005). The findings were confirmed by real-time PCR analysis of gene expression and by immunohistochemistry.

Seven week old male Sprague-Dawley (SD) rats (200-225 grams) were anesthetized with i.p. ketamine (75 mg/kg) and xylazine (10 mg/kg). A severe T10 contusion injury was produced with the New York University (NYU) SCI impactor (10 g×50 mm) (described in detail in Basso, Beattie, and Bresnahan, A Sensitive and Reliable Locomotor Rating Scale for Open Field Testing in Rats J. Neurotrauma 12(1):1-21 (1995)). The control group consisted of age-matched male Sprague Dawley rats subjected to laminectomy only without contusion injury (n=6). For stem cell transplantation (n=11), a midline incision was performed immediately following the contusion injury by a perpendicular stabbing at 5 points along the midline of the lesion site with a 26 gauge needle through the dura to the bottom of the cord. Then, 1,000,000 human dental pulp stem cells (passage 4) suspended in 20 microliters of culture media were injected into the lesion. A further cohort of contused age-matched male Sprague-Dawley rats were subjected to laminectomy and injected with 20 microliters of culture media lacking any human dental pulp stem cells (n=9). Post-injury care for all rats was performed as described in Basso, Beattie, and Bresnahan (BBB). The BBB scale was used to confirm injury severity by rating hind limb functional deficits on day 1, day 2, and then weekly for 10 weeks. The BBB scale is a 21 point assessment of the extent of spinal cord damage based on observable criteria. The scale (0-21 wherein 0 indicates severe spinal cord damage and 21 indicates no spinal cord injury) correlates combinations of rat joint movements, hind limb movements, stepping, forelimb and hind limb coordination, trunk position and stability, paw placement, and tail position with spinal cord damage. Differences in BBB scores were examined using two sample t-tests.

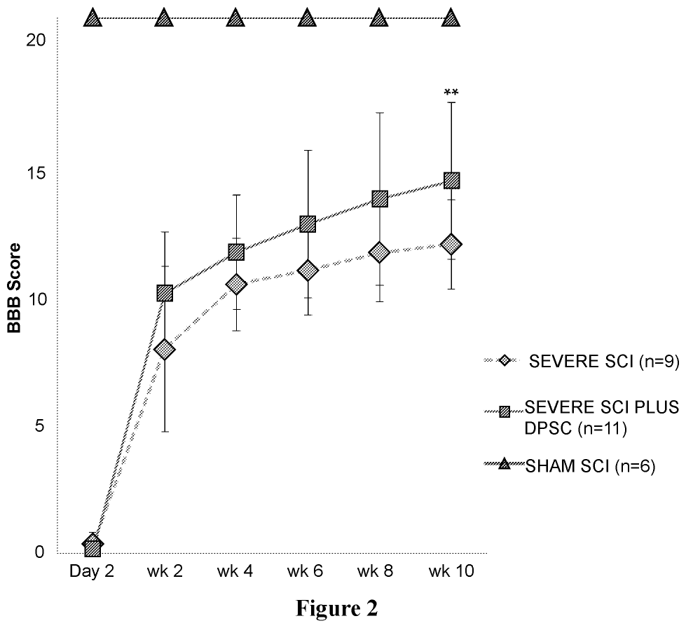

As shown in FIG. 2 the control group (SHAM SCI) without contusion injury exhibited BBB scores of 21, indicating a complete lack of spinal cord injury. FIG. 2 also indicates that the group subjected to contusion injury without treatment with dental pulp stem cells (SEVERE SCI) exhibit very low BBB scores with a rapid partial recovery in the first two weeks followed by a more gradual partial improvement in BBB score. FIG. 2 further indicates that the group subjected to contusion injury and subsequently treated with human dental pulp stem cells (SEVERE SCI PLUS DPSC) exhibit very low BBB scores followed by statistically significant greater rate of recovery and a higher overall BBB score relative to the untreated (SEVERE SCI) cohort. These results indicate that the presence of human dental pulp stem cells improves recovery of rats with severe spinal injury.

### Example 2

### Molecular Characterization of Dental Pulp Stem Cells Cultured Under Neuro-Inductive Conditions

The expression of neural markers such as doublecortin, tubulin, and NeuN was examined in human dental pulp stem cells cultured under a variety of culture conditions and compared with expression of the stem cell marker Oct 3/4 to determine conditions suitable for producing differentiated dental pulp stem cells suitable for use in treatment of contusion spinal cord injury.

Dental pulp stem cells were obtained from third molar teeth and the pulp extracted and grown subject to a variety of processing and growth conditions to produce dental pulp stem cells. A particularly useful method comprises extraction of dental pulp directly from the tooth and culturing the bolus of dental pulp containing the stem cells directly in basal medium, DMEM/F12+15% FBS supplemented with 100 U/mL non-essential amino acids as described above without further treatment. This has the advantage of not introducing any non-exogenous proteins, nucleic acids or other chemical factors to the sample. Cells cultured in this manner for 21 days were harvested, stained with fluorescent conjugated antibodies and subjected to cell sorting.

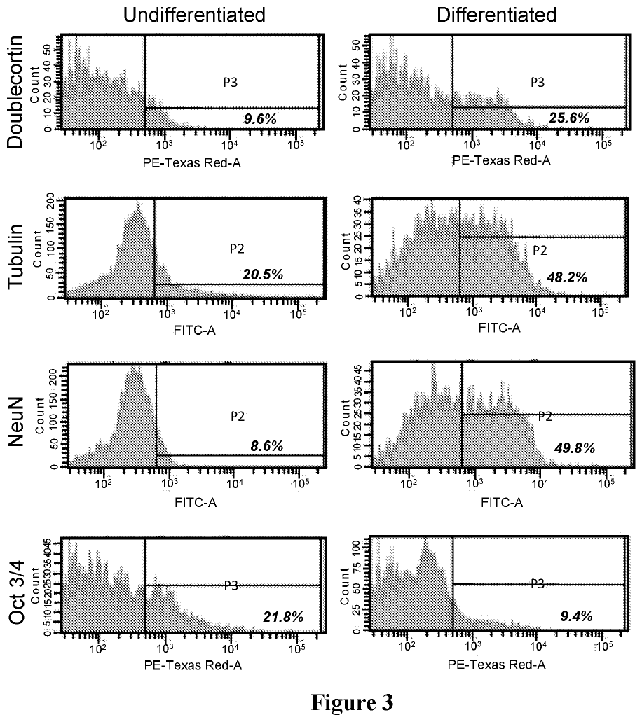

The results of examining culturing dental pulp stem cells in this manner indicate that the stem cells undergo significant neuro-inductive differentiation with the neural markers doublecortin, tubulin, and NeuN increasing approximately 3-, 2.5-, and 6-fold, respectively. Under the same conditions the stem cell marker Oct 3/4 was reduced approximately 2-fold. See FIG. 3.

### Example 3

### Effect of Trauma Induced Inflammation and Autoimmunity on Neural Inductive Capacity of Dental Pulp Stem Cells

This Example examines the effect of inflammatory agents such as TNF-alpha and stem cell induced autoimmunity on the potential efficacy of dental pulp stem cells for treatment of spinal cord injury in vitro.

DPSC were isolated from the teeth of 7 healthy donors extracted during routine dental care. Samples were selected from our tooth biorepository based on the following donor characteristics: absence of medical comorbidities, no active medication use (aside from vitamin supplements), and absence of periodontal disease or tooth decay. The extracted pulp were plated and then cultured in basal medium, DMEM/F12+15% FBS supplemented with 100 U/mL non-essential amino acids (Gibco Life Technologies), 100 U/mL penicillin and streptomycin, and glutamine (Gibco Life Technologies). Outgrowing fibroblast-like cells appeared after 3-4 days and at that point the pulp was replated. The outgrowing cells were passaged after reaching 80% confluence and passage 4 was used for all experiments. Cells were seeded in duplicate with 2×10⁴ cells/well. Neuronal differentiation was induced by replacing DMEM/F12+15% FBS culture media with neuronal differentiation media (NeuroCult NS-A Differentiation Kit-Human, StemCell Technologies Inc., Tukwila, Wash., USA) 48 hours after seeding. Cells were cultured under neuronal induction conditions for up to 21 days with culture media changed twice a week.

Varying concentrations of TNF-alpha (1, 10, 25, and 100 ng/mL, PeproTech, Rocky Hill, N.J., USA), lipoxin A4 (10 and 100 nM, Cayman Chemical, Ann Arbor, Mich., USA) or resolvin D1 (10 and 100 nM, Cayman Chemical, Ann Arbor, Mich., USA) were added to the culture of both differentiated and non-differentiated cells starting 48 hours after seeding. In some experiments cells were treated with both TNF-alpha and lipoxin A4 or resolvin D1.

The metabolic activity of cells was determined by 3-(4,5-dimethylthiazol-2-yl)-2,5-diphenyltetrazolium bromide (MTT) assay at various time points to assess TNF-α toxicity on dental pulp stem cells. Cells were incubated for 4 hours in 5 mg/mL thiazolyl blue tetrazolium bromide (Sigma Aldrich, St. Louis, Mo., USA), the precipitate was suspended with 2-propanol (Sigma Aldrich, St. Louis, Mo., USA), and absorbance was determined in triplicate.

Induction of inflammatory cytokine gene expression was determined by real-time PCR to optimize TNF-α treatment conditions. Total RNA was extracted from cells using Trizol (Life Technologies). 10 μL total RNA was reverse transcribed to cDNA using iScript cDNA Synthesis Kit (Bio-Rad, Hercules, Calif., USA). 1 μL cDNA used as template for real-time PCR amplification reactions using the iQ SYBR Green Supermix (Bio-Rad) in combination with 19 μL of each gene-specific primer, according to manufacturer's instructions. Glyceraldehyde 3-phosphate dehydrogenase (GAPDH) was used as endogenous control. PCR reactions were conducted in duplicate using the following conditions: initial denaturation for 3 min at 95° C. followed by 40 cycles of 15 sec denaturation at 95° C. and 60 sec annealing at 60° C. Relative quantification was used for statistical analyses. The following primers were used:

| | |
|---|---|
| | (SEQ ID NO. 1) |
| IL-6 | Forward: 5'AAATTCGGTACATCCTCGACGG3' |
| | (SEQ ID NO. 2) |
| | Reverse: 5'GGAAGGTTCAGGTTGTTTTCTGC3' |
| | (SEQ ID NO. 3) |
| IL-8 | Forward: 5'ACTGAGAGTGATTGAGAGTGGAC3' |
| | (SEQ ID NO. 4) |
| | Reverse: 5'AACCCTCTGCACCCAGTTTTC3', |
| | (SEQ ID NO. 5) |
| IL-113 | Forward: 5'- AAGGCGGCCAGGATATAACT-3' |
| | (SEQ ID NO. 6) |
| | Reverse: 5'- TACGGCCTAAGGCAGGCAGTTG-3' |
| | (SEQ ID NO: 7) |
| GAPDH | Forward: 5'AGAAAAACCTGCCAAATATGATGAC3' |
| | (SEQ ID NO. 8) |
| | Reverse: 5'TGGGTGTCGCTGTTGAAGTC3' |

Expression of stem cell and neuronal cell markers was determined by flow cytometry. Cells were fixed with 4% paraformaldehyde for 30 minutes, permeabilized with 0.1% Triton X-100 for 10 minutes, and subsequently blocked with TBST in 5% BSA for 20 minutes. Cells were incubated for 1 hour at room temperature with the following primary antibodies against stem cell markers: rabbit anti-CD44 (Abcam, 1:30) and rabbit anti-OCT4 (Abcam, 1:200) and neuronal markers: rabbit anti-nestin (Abcam, 1:100), rabbit anti-doublecortin (Abcam, 1:50), mouse anti-β III-tubulin (Abcam, 1:100), and rabbit anti-NeuN (Abcam, 1:100). Fluorescence was achieved by incubating with secondary antibodies (Life Tech, Carlsbad, Calif., goat anti-rabbit IgG or goat anti-mouse IgG, dilution 1:500) for 1 hour at room temperature in a dark room. Cells were then subjected to flow cytometry and the percentage of cells expressing each marker was calculated.

Immunocytochemistry was performed on cells according to standard protocols. Briefly, cells were cultured on glass cover slips under varying conditions, washed 3×10 minutes in 1×PBS+0.1% (vol/vol) Tween-20, fixed in 4% paraformaldehyde (Sigma-Aldrich, St. Louis, Mo., USA) in PBS for 10 minutes, permeabilized with 0.1% Triton X-100 (Sigma-Aldrich, St. Louis, Mo., USA) in PBS for 10 minutes, and washed 3×10 minutes in ice-cold PBS. The cells were then incubated with the following primary antibodies (Nestin-1:200, Tubulin-1:100 and NeuN 1:500, Abcam, Cambridge, Mass., USA) in PBS with 3% bovine serum albumin (BSA, Sigma-Aldrich, St. Louis, Mo., USA) for 1 hour in a humidified chamber at 4° C. The cells were then washed 3× for 10 minutes in 1×PBS+0.1% (vol/vol) Tween-20 and incubated for 1 hour at room temperature with the biotinylated secondary antibodies (100 μl/coverslip, diluted 1:250 to 1:750 in blocking buffer, Life Tech, Carlsbad, Calif., goat anti-rabbit IgG or goat anti-mouse IgG) in a humidified chamber. Finally, cells were washed 3× for 5 minutes in 1×PBS, incubated for 1 min with 1 mg/mL 4′6′-Diamidin-2-phenylindol (DAPI, Vector Laboratories, Burlingame, Calif.), rinsed in 1×PBS, and the coverslips were mounted with Vectashield mounting medium and sealed. Images were acquired a Carl Zeiss Axioplan fluorescence microscope (LSM 410, Zeiss, Jena, Germany).

Each analysis was performed in duplicate using cells from all donors. Median and interquartile ranges were calculated for each condition. Mann-Whitney tests were used for all statistical analyses; p<0.05 was considered significant.

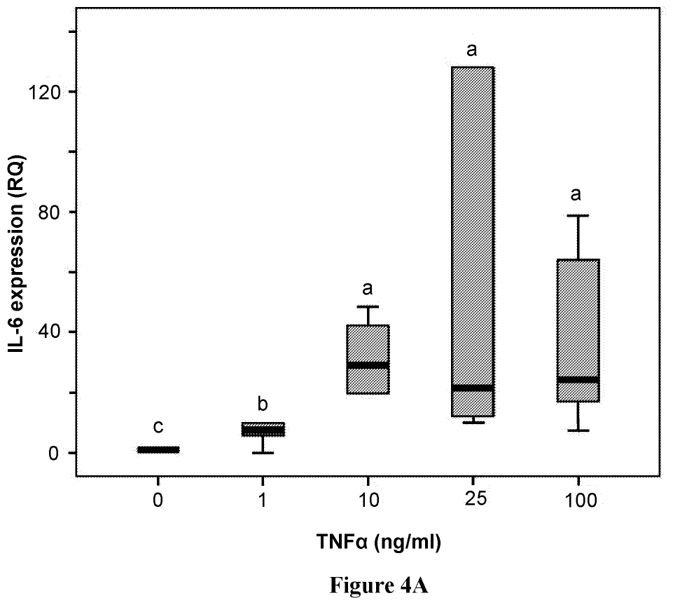

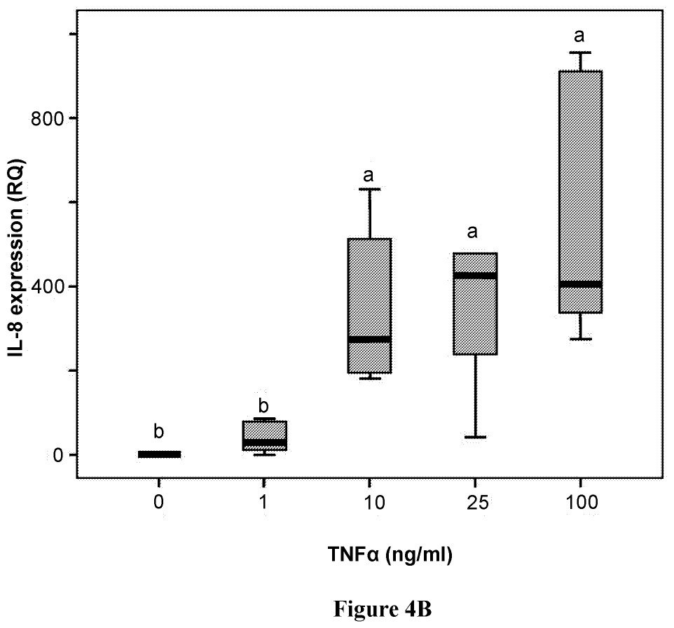

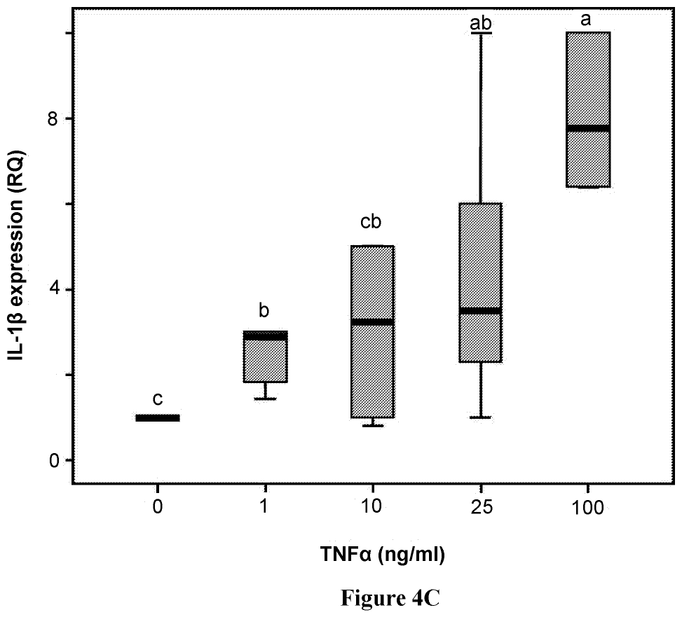

TNF-alpha treatment of DPSC was optimized by assessing changes in metabolic activity and inflammatory cytokine production. Neural induction resulted in a significant reduction in metabolic activity of DPSC at both 2 and 10 days. Neural induction in the presence of either 25 ng/ml or 100 ng/ml TNF-alpha resulted in significantly greater metabolic activity at 10 days compared to cells induced in the absence of TNF-alpha. Similarly, all doses of TNF-alpha stimulated significant increases in DPSC expression of the inflammatory cytokines IL-6, IL-8, and IL-1β after 24 hours of treatment (FIGS. 4A, 4B, and 4C, respectively). Based on these results, the concentration of 25 ng/ml was chosen for all subsequent experiments.

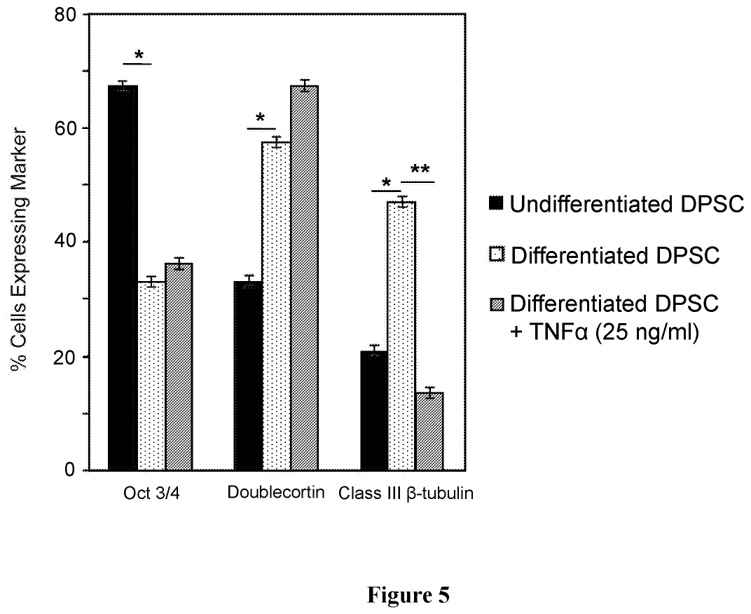

At 21 days of neuronal induction, there was a significant reduction (52% reduction, p<0.001) in the percentage of cells expressing the stem cell marker OCT 3/4. Concomitantly, significantly more cells expressed the early neuronal marker doublecortin (3% increase, p<0.002), and the intermediate neuronal marker β III-tubulin (124% increase, p<0.01). These findings were confirmed by real-time PCR analysis of gene expression (data not shown) and by immunocytochemistry (FIG. 5).

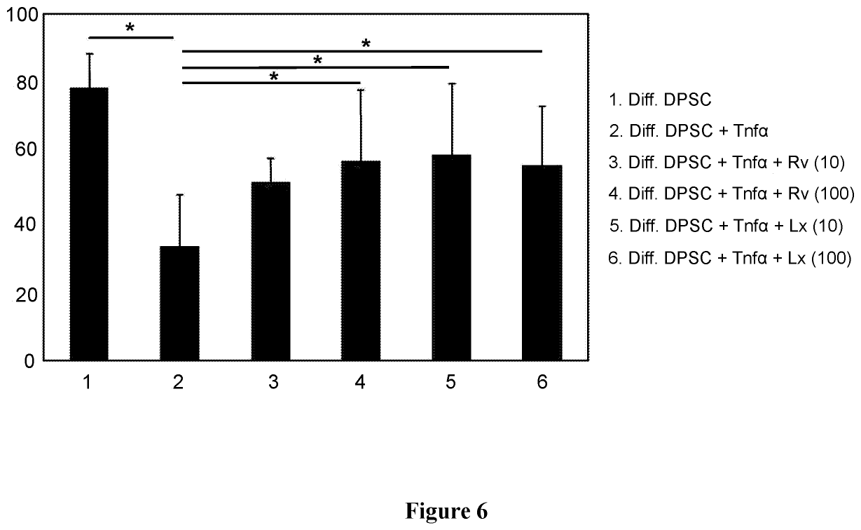

We tested the effect of 25 ng/ml TNF-alpha on DPSC neuronal differentiation. We found no difference in the expression of stem cell markers (CD44, OCT 3/4) or the early neuronal marker nestin by flow cytometry at 21 days of differentiation in the presence of 25 ng/ml TNF-alpha compared to cells differentiated in the absence of TNF-alpha. However, there was a non-significant increase in cells expressing the early marker doublecortin and a dramatic reduction in the percentage of differentiated cells expressing the intermediate neuronal marker 13 III-tubulin (70% decrease, p<0.004). These findings are consistent with failure to progress to late neuronal differentiation in the presence of 25 ng/ml TNF-alpha (FIGS. 4 and 6). These findings were confirmed by real-time PCR analysis of gene expression (data not shown) and by immunocytochemistry.

We tested the effect of lipoxin A4 or resolvin D1 to mitigate TNF-alpha-induced suppression of DPSC neuronal differentiation. Results presented in FIG. 6. We confirmed that 25 ng/ml TNF-alpha significantly reduced β III-tubulin expression (FIG. 3, 59% decrease, p=0.0002). This reduction was partially blocked by treatment with either resolvin D1 (100 nM) or lipoxin A4 (10 nM). We found a significant 78% increase in β III-tubulin expression in cells undergoing neural induction in the presence of both TNF-alpha and 100 ng/ml resolvin D1 (32% for 25 ng/ml TNF-alpha vs 57% for 25 ng/ml TNF-alpha+resolvin D1, p=0.02). Similarly, we found a significant 84% increase in β III-tubulin expression in cells undergoing neural induction in the presence of both TNF-alpha and 10 nM lipoxin A4 (32% for 25 ng/ml TNF-alpha vs 59% for 25 ng/ml TNF-alpha+lipoxin A4, p=0.02). Results were similar with lipoxin A4 at the 100 nM dose (p=0.03) and approached significance with resolving D1 10 nM (p=0.09). We observed no reduction or improvement in neuronal differentiation when DPSC cells underwent neuronal induction with the addition of either resolvin D1 or lipoxin A4 at either dose (data not shown).

We found that dental pulp stem cells fail to progress to late neuronal differentiation when cultured in the presence of TNF-alpha. We confirmed that TNF-alpha treatment was not toxic to DPSC, as demonstrated by succinic dehydrogenase metabolic activity. Our findings of reduced metabolic activity with neural induction is consistent with other reports, suggesting a metabolic switch from proliferation to differentiation (Spath, et al., Explant-derived human dental pulp stem cells enhance differentiation and proliferation potentials Journal of Cellular and Molecular Medicine 14(6B):1635-44 (2010), Ferro, et al., Dental pulp stem cells differentiation reveals new insights in Oct4A dynamics PLoS One 7(7):e41774 2012, Insco, et al., A self-limiting switch based on translational control regulates the transition from proliferation to differentiation in an adult stem cell lineage Cell Stem Cell 11(5):689-700 (2012). TNF-alpha treatment resulted in significant increase in metabolic activity at 10 days of neural induction, suggesting inflammatory conditions promoted proliferation and suppressed neural differentiation. This finding was confirmed by both flow cytometry and immunocytochemistry. Since inflammation is an inherent response following SCI, therapies using stem cell transplantation must consider the microenvironment of inflammatory cytokines and mediators in which the cells must proliferate and differentiate. Transplanting dental pulp stem cells in conjunct with administering anti-inflammation agents into SCI may enhance therapeutic benefits by enabling complete dental pulp stem cells differentiation and cell replacement.

### Example 4

### Surgical Transplantation of Autologous Dental Pulp Stem Cells to Treat Acute Thoracic Motor Complete SCI

This Example describes a method of treating a human spinal cord injury using autologous dental pulp stem cells.

Population:

Patients in the trial will have spinal cord injury categorized as T3-10 ASIA Impairment Scale (AIS) A or B, due to traumatic SCI with a single cord lesion on MRI, male or female, age 18-50, with acute injury (within 3 months post-injury).

Method for Extracting Autologous Dental Pulp Stem Cells:

Dental pulp stem cells are isolated during a bedside, tooth-preserving root canal procedure from a healthy permanent tooth, such as a molar, (without caries or sign of decay) within 2 weeks of injury.

Cell Preparation:

Pulp is extracted from the tooth by cutting the tooth longitudinally using a circular saw. Freshly extracted pulp containing dental pulp stem cells is then placed in a culture dish on DMEM/F12 medium/FBS without any enzymatic treatment. Approximately one week later, the first outgrowing fibroblast-like cells are expected to be observed. At that point, the pulp including the outgrowing fibroblast-like cells is transferred to a new dish without use of any enzymatic treatment. After each transfer the pulp yields an increasing number of outgrowing fibroblast-like cells approximately every three or four days. This generates sufficient numbers of cells for transplantation within about 14-21 days. To prepare dental pulp stem cells for injection the cells (passaged 0-4 times) are suspended in sterile normal saline at a concentration of 5×10⁶ cells/ml and loaded into a needle and transported to the surgical team.

Intervention:

A single dose of autologous dental pulp stem cells is administered 45-60 days after injury. Cord untethering and expansion duraplasty is performed to treat arachnoid scarring prior to injection. Cells are then be injected directly into the exposed spinal cord under general anesthesia. The cord is exposed by posterior laminectomy. The lesion is directly visualized and confirmed by intraoperative ultrasound. Cells are injected (5×10⁶ cells/cm³ per lesion volume suspended in 1 ml) at the caudal aspect of the lesion epicenter.

1. A pharmaceutical formulation comprising dental pulp stem cells and a pharmaceutically acceptable carrier, wherein the dental pulp stem cells are derived from dental pulp from a permanent tooth.

2. The pharmaceutical formulation of claim 1, wherein the dental pulp has not been exposed to an exogenous proteolytic enzyme.

3. The pharmaceutical formulation of claim 1, wherein the dental pulp stem cells do not comprise exogenous DNA.

4. The pharmaceutical formulation of claim 1, comprising at least 10⁶, 2×10⁶, 3×10⁶, 4×10⁶, 5×10⁶, 6×10⁶, 7×10⁶, 8×10⁶, 9×10⁶, or 10⁷ dental pulp stem cells.

5-7. (canceled)

8. A method of preparing dental pulp stem cells, comprising:
(a) culturing dental pulp extracted from a permanent tooth, in a first culture medium until the dental pulp yields outgrowing dental pulp stem cells;
(b) culturing the dental pulp from the first culture medium in a second culture medium until the dental pulp yields outgrowing dental pulp stem cells;
(c) optionally, culturing the dental pulp of step (b) in a third culture medium until the dental pulp yields outgrowing dental pulp stem cells; and
(d) expanding the dental pulp stem cells of the first, second, and optional third culture medium until the dental pulp stem cells reach about 50-70% confluence, and optionally passaging and expanding the dental pulp stem cells from each culture medium up to three times, wherein the dental pulp is not exposed to an exogenous proteolytic enzyme and the dental pulp stem cells lack exogenous DNA.

9. The method of claim 8, wherein each culturing step is performed for 3-4 days.

10-11. (canceled)

12. The method of claim 8, further comprising after the final culturing step, suspending the dental pulp stem cells in a pharmaceutically acceptable carrier.

13. The method of claim 12, further comprising loading the suspended dental pulp stem cells into an injection device.

14. The method of claim 13, wherein the injection device is a syringe.

15. The method of claim 8, wherein the tooth is a molar.

16. A method of treating a spinal cord injury comprising a spinal cord lesion in a patient in need thereof, comprising:
administering dental pulp stem cells to the patient, wherein the dental pulp stem cells are from dental pulp from a permanent tooth extracted from the patient, wherein the dental pulp has not been exposed to an exogenous proteolytic enzyme, and wherein the dental pulp stem cells do not comprise exogenous DNA.

17. The method of claim 16, wherein the tooth is a molar.

18. The method of claim 16, wherein the dental pulp stem cells are administered 45-60 days after the patient suffers the spinal cord lesion.

19. (canceled)

20. The method of claim 16, wherein the administering step comprises directly injecting the dental pulp stem cells into the spinal cord of the patient.

21. (canceled)

22. The method of claim 20, wherein the spinal cord is exposed.

23. The method of claim 22, wherein the spinal cord is exposed by performing a posterior laminectomy.

24. The method of claim 16, further comprising before the administering step, performing cord untethering and expansion duraplasty on the patient.

25. The method of claim 16, wherein the patient's spinal cord injury is categorized as T3-10 ASIA Impairment Scale (AIS) A or B, due to traumatic spinal cord injury with a single spinal cord lesion.

26. The method of claim 16, wherein the patient suffers the spinal cord injury within 3 months to 1 year of the administering step, or more than 1 year before the administering step.

27. The method of claim 26, wherein the spinal cord injury is a cervical or thoracic motor complete injury categorized as MS A or B at level C4-C8, or is a motor incomplete injury categorized as AIS C or D.

28-33. (canceled)
